(19) United States
(12) Patent Application Publication
(10) Pub. No.: US 2017/0368087 A1
(43) Pub. Date: Dec. 28, 2017

(54) PHARMACEUTICAL COMPOSITION COMPRISING TOPIRAMATE

(71) Applicant: INNOVACO PHARMACEUTICALS, INC., Beijing (CN)

(72) Inventors: William S. WEI, Beijing (CN); Herling UANG, Beijing (CN)

(21) Appl. No.: 15/540,181

(22) PCT Filed: Dec. 15, 2015
(86) PCT No.: PCT/CN2015/097451

§ 371 Date: Jun. 27, 2017

(30) Foreign Application Priority Data
Dec. 27, 2014 (CN) 201410832203.7

(51) Int. Cl. A61K 31/7048; A61K 47/36; A61K 47/32; A61K 47/14; A61K 47/12; A61K 47/10; A61K 47/02; A61K 47/38; A61K 9/48

(52) U.S. Cl. CPC A61K 31/7048; A61K 47/38; A61K 47/36; A61K 47/32; A61K 9/4808; A61K 47/12; A61K 47/10; A61K 47/02; A61K 47/14

(57) ABSTRACT

The present invention relates to a pharmaceutical composition comprising topiramate, and more particularly, to a solid pharmaceutical composition comprising topiramate, a cellulose derivative, a lipid and optional pharmaceutical excipients, wherein the weight ratio of topiramate to the cellulose derivative is 100:10 to 100. The pharmaceutical compositions of the present invention have excellent formulation properties as well. The pharmaceutical compositions of the present invention can be used for monotherapy for patients who are newly diagnosed with epilepsy, or patients who have been previously treated with combination therapy and now for monotherapy. They can also be used for the add-on treatment of partial seizures in adults and children aged 2 to 16 years.

PCT Published Abstract

A solid pharmaceutical composition comprising topiramate and a process for the preparation thereof comprising:a topiramate, a cellulose derivative, a lipid and optionally a pharmaceutical adjuvant, wherein the weight ratio of topiramate to the cellulose derivative is 100:10˜100.

### FIELD OF THE INVENTION

This invention pertains to the field of pharmaceutical technology and relates to a pharmaceutical composition comprising topiramate.

### BACKGROUND

Epilepsy is a disease of the nervous system which is caused by brain dysfunction due to excessive discharge of the nerve cells in the brain. It is estimated that the incidence rate of epilepsy is from about 0.3% to 0.5% globally. The morbidity rate is from about 5 to 10 people per 1000 people. Epilepsy is a serious threat to people's health and affects their daily lives.

Topiramate is a new type of antiepileptic drug and its chemical structure relates to amino-sulfamatemonosaccharide. Topiramate was first developed by Johnson & Johnson Company, Inc. USA. It was marketed under the brand name of Topamax in the UK in 1995. Based on in-vitro studies of neurons in electrophysiological and biochemical experiments, it was found that there were three mechanisms of antiepileptic action. Firstly, topiramate blocks the neuron's depolarization, which indicates that it can block sodium channels. Secondly, topiramate can increase the frequency of activation of γ-aminobutyrate (GABA) receptors by GABA and enhance the ability of influx of chloride ions which indicates that topiramate can enhance the role of inhibitory central neurotransmitters. Thirdly, topiramate can reduce the activity of glutamate AMPA receptors which indicates that topiramate can reduce the effect of excitatory central neurotransmitters.

The chemical name of topiramate is 2,3:4,5-Di-O-isopropylidene-β-D-fructo-pyranose sulfamate. The molecular formula is C₁₂H₂₁NO₈S. Its molecular weight is 339.4. The chemical formula is:

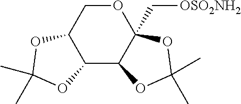

Topiramate is a white crystalline powder with a bitter taste. It is freely soluble in acetone, dimethyl sulfoxide, ethanol, and alkaline solutions containing sodium hydroxide or sodium phosphate. Its solubility in water is approximately 9.8 mg/mL at room temperature.

Topiramate is sensitive to heat and humidity. When exposed to moisture or heat, it can lead to the degradation of the topiramate in a solid dosage form. Topiramate degradation can be easily detected by the changing in physical appearance (tablet color changing from white to brown or black) and the formation of sulfate ions and organic degradation compounds. This degradation can be also detected by analytical testing methods such as HPLC.

In order to improve the stability of topiramate and to prevent the degradation of its active ingredient, WO01/89445 discloses topiramate in a blister package without desiccant. The blister pack comprises of a disc-like sheet for placing a pre-dried chamber with the topiramate and a cover sheet for sealing the disc-like sheet. The currently marketed topiramate tablets are mostly packaged in this type of blister pack. But this type of packaging is relatively expensive and the operation process is cumbersome. CN1726011 discloses a double or multiphase topiramate tablet and its preparation method. The tablet has one phase containing topiramate and another phase containing hygroscopic gum material which is selected from alginates, gum arabic or xanthan gum. This preparation is cumbersome, is easy to laminate, and has a low dissolution rate. WO2006/097946 discloses topiramate tablet preparation containing 5%-35% (w/w) of topiramate and 25%-70% (w/w) of spray-dried mannitol granules. The tablet is prepared by by direct compression. The tablet is easy to get capping and has poor content uniformity.

CN103417501A (201210162377.8) discloses a topiramate pharmaceutical composition containing pregelatinized starch to increase the stability of topiramate which in turn ensures the quality and safety of the composition. Pregelatinized starch also acts as a binder to ensure the hardness and low friability reducing the amount of excipients and cost. This invention also relates to a method of preparing topiramate pharmaceutical composition which includes a dry granulation process. The process is simple; granules have uniform particle size distribution after drying; and it is convenient for storage and transportation and suitable for commercial manufacturing. However, the dry granulation process is not suitable for a large-scale production. Additionally, the use of pregelatinized starch and disintegrating agent in the composition causes a very fast dissolution rate which can not achieve a delayed release effect.

Thus, to obtain a topiramate formulation with stable chemical properties and/or desired sustained release effect is highly desirable.

### SUMMARY OF THE INVENTION

The purpose of this invention is to provide a solid pharmaceutical composition of topiramate with chemical stability and/or a designable extended release profile. It has been surprisingly found that the pharmaceutical compositions of the present invention have achieved the characteristics of all the above objections. Therefore, the present invention is accomplished by the above discovery.

Thus, this invention in the first aspect provides solid pharmaceutical compositions containing topiramate, cellulose derivatives, lipids, and optionally other pharmaceutical excipients.

According to any example of the solid pharmaceutical compositions described in the first aspect of the present invention, the weight ratio of topiramate to cellulose derivative is 100:10˜100.

According to any example of the solid pharmaceutical compositions described in the first aspect of the present invention, the weight ratio of topiramate to cellulose derivative is 100:15˜75.

According to any example of the solid pharmaceutical compositions described in the first aspect of the present invention, the weight ratio of topiramate to cellulose derivative is 100:20˜50.

According to any example of the solid pharmaceutical compositions described in the first aspect of the present invention, the cellulose derivative can be selected from the group consisting of: methyl cellulose, ethyl cellulose, sodium carboxymethyl cellulose, hydroxypropyl cellulose, hydroxyethyl cellulose, hydroxypropylmethylcellulose and their combination. In one example, the cellulose derivative is hydroxypropyl methylcellulose.

According to any example of the solid pharmaceutical compositions described in the first aspect of the present invention the weight ratio of topiramate to lipid substance is 100:10˜100.

According to any example of the solid pharmaceutical compositions described in the first aspect of the present invention, the weight ratio of topiramate to lipid substance is 100:15˜75.

According to any example of the solid pharmaceutical compositions described in the first aspect of the present invention, the weight ratio of topiramate to lipid substance is 100:20˜50.

According to any example of the solid pharmaceutical compositions described in the first aspect of the present invention, the lipid substance mentioned above is a type of a strong lipophilic substance, including but not limited to: C16-C22 fatty acids, carnauba wax, C16-C22 fatty acid esters of glycerol, C16-C22 alkyl alcohol, bee wax, synthetic wax, hydrogenated vegetable oil and their combination. In one of the examples, the lipid substance can be selected from the group consisting of: C16-C22 fatty acids, carnauba wax, C16-C22 fatty acid glyceride, C16-C22 alkyl alcohol and any combination thereof. In one of the examples, the lipid substance is C16-C22 fatty acid esters of glycerol. In one of the examples, the lipid substance is glyceryl behenate. In one of the examples, glyceryl behenate can be selected from the group consisting of: behenic acid monoglyceride, diglyceride behenic acid, behenic acid triglyceride and any combination thereof.

The solid pharmaceutical composition according to any example of the solid pharmaceutical compositions described in the first aspect of the present invention has a topiramate content of from 1 to 99%, from 2 to 75%, or from 5 to 50% based on the total weight of the solid pharmaceutical composition. Because the present invention uses a lipid substance and a cellulose derivatives in combination with topiramate to form a solid pharmaceutical composition, excellent performance of the composition can be achieved. Thus, in addition to the lipid and the cellulose derivatives and topiramate, the amount of other pharmaceutical excipients is not particularly limited. These excipients are usually added in an amount suitable to impart a suitable dosage form of the composition. For example, when preparing tablets or capsules, pharmaceutical excipients may be added appropriately to lower the overall weight of the dosage form. For example, when the granules are prepared, the total weight of the dosage form may be higher, as well as the weight of the pharmaceuticals excipients.

According to any example of the solid pharmaceutical compositions described in the first aspect of the present invention, the pharmaceutical excipient is one or more selected from the group consisting of a filler, a disintegrant, a binder and a lubricant. In view of the present invention, it was surprisingly discovered that when topiramate is mixed with the lipid and the cellulose derivatives, the mixture enhances the stability of topiramate, i.e. the contribution of present invention to the current technology is the discovery of such mentioned combination. Thus, the combination may or may not include other pharmaceutical excipients.

According to any example of the solid pharmaceutical compositions described in the first aspect of the present invention, the fillers (generally also referred to as a diluents) include, but not limited to, starch or its derivatives such as corn starch, pregelatinized starch, modified starch, etc., cellulose or derivatives thereof such as microcrystalline cellulose, ethyl cellulose, methylcellulose etc; saccharides such as glucose, sucrose, lactose, mannitol, sorbitol; neutral minerals such as calcium carbonate, calcium hydrogen phosphate, and combinations thereof. As used herein, the term diluent or filler is defined as an inert material used for making up the weight and/or size of the pharmaceutical composition in the form of a substance in the composition or a mixture of compounds. Preferably, a diluent or filler is added when the amount of the active ingredient and other excipients are not enough to create a desired tablet size. The amount of the diluent or filler for the pharmaceutical composition according to the present invention may be determined according to conventional methods by a person skilled in the art, particularly after the amount of other excipients such as disintegrating agents, binders, lubricants etc. then the amount of diluents and/or fillers can be determined based on the size requirement.

According to any example of the solid pharmaceutical compositions described in the first aspect of the present invention, the disintegrant comprises, but is not limited to, cross-linked polyethylene polypyrrolidone, sodium starch glycolate, croscarmellose sodium, Hydroxypropylcellulose etc, and combinations thereof. Further, the pharmaceutical composition comprises 0 to 10% disintegrant, 0 to 8% disintegrant, or 0 to 5% disintegrant relative to the total percentage weight of the pharmaceutical composition, The use, not use, or use less quantity of disintegrants are also known to the person skilled in the art, such as erosion-type matrix sustained-release tablets. The solid pharmaceutical compositions of the present invention may in some cases have no disintegrants. For example, when the dosage form is capsule, the disintegrants may not be added to the solid pharmaceutical compositions of the present invention.

According to any example of the solid pharmaceutical compositions described in the first aspect of the present invention, the binder comprises, but is not limited to, polyethylene glycol, starch, polyvinylpyrrolidone, hydroxypropylmethylcellulose etc., and combinations thereof. Water can be used as a wetting agent in the present invention, and can also be used as a potential binder in wet granulation process because many excipients in solid pharmaceutical formulations have a certain degree of adhesiveness and water can be removed from the final product. In addition, many excipients in solid pharmaceutical formulations have adhesiveness and allow direct compression or encapsulation. Therefore, a binder may or may not be added to the pharmaceutical composition of the present invention. Even with the wet granulation technology, a binder may not be added. If a binder is added, the amount of the binder used is from 0.1 to 10%, 0.2 to 5%, or 0.5 to 2.5% relative to the total percentage weight of the pharmaceutical composition, or it may also be used based on the experience of a person skilled in the art.

According to any example of the solid pharmaceutical compositions described in the first aspect of the present invention, the lubricant (including a glidant) functions as the powder material which can assist powders to form a dosage form. For example, when a capsule is prepared, the powder material can be filled uniformly into the capsule shell. For example, when the tablet is compressed, the powder material can be filled uniformly into the tooling mold of the tablet press and compressed without stickingness. Examples of lubricants include, but are not limited to, magnesium stearate, calcium stearate, talc, starch, stearic acid, colloidal silica, polyethylene glycol, etc. If added, the amount of lubricant used is from 0.1 to 10%, 0.2 to 5%, or 0.2 to 2% relative to the total percentage weight of the pharmaceutical composition, or it may also be used based on the experience of a person skilled in the art.

According to any example of the solid pharmaceutical compositions described in the first aspect of the present invention, the pharmaceutical dosage form is selected from the group consisting of tablets, capsules, mini-tablet in capsules, etc. The term of mini-tablet in capsules refers to a form of formulation in which the small size tablets (e.g., 30 to 100 mg per tablet, for example, tablets each weighing 30 to 50 mg) are encapsulated into a hard shell capsule.

According to any example of the solid pharmaceutical compositions described in the first aspect of the present invention, the pharmaceutical dosage form is uncoated tablet or coated tablet with a coating material.

According to any example of the solid pharmaceutical compositions described in the first aspect of the present invention, the pharmaceutical dosage form is a coated tablet. Further, the coated tablet comprises a coating material of 1% to 6%, or 2% to 5% by percentage weight gain based on the total weight of the tablet. In one embodiment, the coating material is selected from the group consisting of ethyl cellulose, hydroxypropylmethylcellulose and methacrylic acid-alkyl acrylate copolymers. In one embodiment, the coating material is an aqueous dispersion of hydroxypropylmethylcellulose. In the other embodiment, the coating material is Opadry®, which is an aqueous dispersion of hydroxypropylmethylcellulose. Further, the coating material is selected from the group consisting of Opadry® 85F20694, Opadry® 85F32004, Opadry® 85F23452 and Opadry®85F18422.

According to any example of the solid pharmaceutical compositions described in the first aspect of the present invention comprises:

Topiramate: 100 parts by weight,

cellulose derivatives: 10 to 100 parts by weight,

lipid substances: 10 to 100 parts by weight,

pharmaceutical excipients: 0 to 500 parts by weight.

The solid pharmaceutical composition according to any example of the solid pharmaceutical compositions described in the first aspect of the present invention comprises:

Topiramate: 100 parts by weight,

cellulose derivative: 15 to 75 parts by weight,

lipid substances: 15 to 75 parts by weight,

pharmaceutical excipients: 0 to 250 parts by weight.

According to any example of the solid pharmaceutical compositions described in the first aspect of the present invention comprises:

Topiramate: 100 parts by weight,

cellulose derivatives: 20 to 50 parts by weight,

lipid substances: 20 to 50 parts by weight,

pharmaceutical excipients: 0 to 200 parts by weight.

According to any example of the solid pharmaceutical compositions described in the first aspect of the present invention, the pharmaceutical composition is placed at a 40° C. condition for 5 months, wherein the impurity of 2,3:4,5-di-O-(1-methylethylidene)-β-D-fructose fructose by weight (%) before and after high temperature treatment, is less than 100%, especially less than 80%, especially less than 70%: for example, from 40 to 100%, from 40 to 80%, or from 40 to 70%.

Furthermore, the second aspect of the present invention provides a method for preparing a solid pharmaceutical composition, (such as any example of the solid pharmaceutical compositions described in the first aspect of the present invention), comprising the steps of as follows:

(1) Each of the materials were pulverized to pass through a 60 mesh sieve, the amount of topiramate and cellulose derivatives, and lipid substances are mixed thoroughly to obtain a powder mixture;

(2) The solid pharmaceutical preparation is prepared by mixing the powder mixture in step (1) with an optional pharmaceutical excipient and making into suitable solid dosage form according to conventional solid pharmaceutical preparation.

According to any example of the methods described in the second aspect of the present invention, the solid pharmaceutical preparation prepared in step (2) is selected from the group consisting of tablets, capsules and minitabletin capsules.

According to any example of the methods described in the second aspect of the present invention wherein in the step (2), when the solid pharmaceutical preparation is prepared, the granules can be prepared by a wet granulation process or a dry granulation process, then compressed into tablets, or encapsulated into capsules, or encapsulated as the minitablets into capsules.

According to any example of the methods described in the second aspect of the present invention wherein in the step (2), when the solid pharmaceutical preparation is prepared, granulation step may not be necessary. The powder mixture can be compressed directly to tablets, encapsulated, or encapsulated as microtablets in capsules without the granulation steps.

According to any example of the methods described in the second aspect of the present invention, the pharmaceutical composition comprises of topiramate, cellulose derivative, lipid, and pharmaceutical excipients of choice.

According to any example of the methods described in the second aspect of the present invention, the weight ratio of topiramate to the cellulose derivative in the pharmaceutical composition is from 100:10 to 100.

According to any example of the methods described in the second aspect of the present invention, the weight ratio of topiramate to the cellulose derivative in the pharmaceutical composition is 100:15 to 75.

According to any example of the methods described in the second aspect of the present invention, the weight ratio of topiramate to the cellulose derivative in the pharmaceutical composition is 100:20 to 50.

According to any example of the methods described in the second aspect of the present invention, the cellulose derivative described in the pharmaceutical composition is selected from the group consisting of methylcellulose, ethyl cellulose, sodium carboxymethylcellulose, propylcellulose, hydroxyethylcellulose, hydroxypropylmethylcellulose and combinations thereof. In one embodiment, the cellulose derivative is hydroxypropylmethylcellulose.

According to any example of the methods described in the second aspect of the present invention, the weight ratio of topiramate to the lipid substance in the pharmaceutical composition is 100:10 to 100.

According to any example of the methods described in the second aspect of the present invention, the weight ratio of topiramate to the lipid substance in the pharmaceutical composition is 100:15 to 75.

According to any example of the methods described in the second aspect of the present invention, the weight ratio of topiramate to the lipid substance in the pharmaceutical composition is 100:20 to 50.

According to any example of the methods described in the second aspect of the present invention, the lipid substance in the pharmaceutical composition is a lipophilic substance which include, but is not limited to, C16-C22 fatty acid, Brazil Palm wax, C16-C22 fatty acid glycerides, C16-C22 alkyl alcohols, beeswax, synthetic waxes, hydrogenated vegetable oils and mixtures thereof. In one embodiment, the lipid is selected from the group consisting of: C16-C22 fatty acids, carnauba waxes, C16-C22 fatty acid glycerides, C16-C22 alkyl alcohols and mixtures thereof. In one embodiment, the lipid is a C16-C22 fatty acid glyceride. In one embodiment, the lipid is glyceryl behenate. In one embodiment, the behenate is selected from the group consisting of monoglycerides, behenic acid diglycerides, behenic acid triglycerides, and mixtures thereof.

According to any example of the methods described in the second aspect of the present invention, the topiramate comprises from 1 to 99%, such as from 2 to 75%, such as from 5 to 50% of the total weight of the pharmaceutical composition. The present invention uses a lipid substance and a cellulose derivative in combination with topiramate to form a solid pharmaceutical composition with excellent property. Thus, in addition to the combination of the lipid and the cellulose derivative and the topiramate, the amount of the other pharmaceutical excipients are not particularly limited, usually, to make the desirable weight of certain dosage form. For example, when formulating tablets or capsules, because the amount of the lipid substance and cellulose derivative combination is fixed, the other pharmaceutical excipients can be adjusted due to the final total weight of the dosage form. For example, when prepare for granules, because the weight of the dosage form is increased, the amount of other pharmaceutical excipients will be increased.

According to any example of the methods described in the second aspect of the present invention, one or more excipients used in the described drug combination are selected from the following groups: filler, disintegrant, binder, lubricant. The surprising discovery of current invention is that the combination of topiramate, lipid, and the cellulose derivatives can provide an excellent chemical stability for topiramate. Because the particular contribution of the present invention to the prior art is the discovery of such a special combination, the described pharmaceutical excipients may or may not be included in the composition of the present invention.

According to any example of the methods described in the second aspect of the present invention, the filler (usually also referred to as diluent) in the solid pharmaceutical composition include but not limited to: starch or its derivatives such as corn starch, pregelatinized starch, modified starch, etc.; cellulose or its derivatives such as microcrystalline cellulose, ethyl cellulose, methyl cellulose, etc.; carbohydrates such as glucose, sucrose, lactose, mannitol, sorbitol; A neutralized minerals such as calcium carbonate, calcium hydrogen phosphate,etc and its combinations. As used herein, the term “diluent” or “filler” is defined as an inert material to increase the weight and/or size of the pharmaceutical compositions, which exists in the form of a substance or a mixture of compounds in the composition. Preferably, a diluent or filler is added when the amount of the active ingredient and other excipients is too small to obtain a tablet of a suitable size. The percentage weight of the diluent or filler necessary for the pharmaceutical composition, according to the present invention, may be determined by conventional methods well known to a person skilled in the art, especially a moderate amount of the diluent or filler will be added due to the requirement of the product size after the amount of other excipients such as disintegrating agents, binders, lubricants are confirmed.

According to any example of the methods described in the second aspect of the present invention, the disintegrant used in the described solid pharmaceutical composition includes but not limited to: cross-linked polyethylene polypyrrolidone, sodium starch glycolate, croscarmellose sodium, low-substituted hydroxypropylcellulose and their combinations. Furthermore, the pharmaceutical composition comprises 0 to 10%, from 0 to 8% or 0 to 5% of disintegrant relative to the total weight of the composition/The amount of the disintegrant also can be based on the experience of a technical personnel in the field to use no or less amount of disintegrant in drug product, such as dissolved-matrix type sustained-release tablets made for sustained-release purposes. The solid pharmaceutical compositions in the present invention may have no disintegrating properties in some cases. For example in some cases, for capsules formulation, the disintegrants may not be added to the solid pharmaceutical compositions of the present invention.

According to any example of the methods described in the second aspect of the present invention, the binder in the described solid pharmaceutical composition includes. but not limited to: polyethylene glycol, starch, polyvinylpyrrolidone, hydroxypropylmethylcellulose, etc and their combinations. Water as a wetting agent in the present invention can also be used as a potential binder because many excipients in solid pharmaceutical formulations have a certain degree of adhesiveness and can be used during wet granulation, although the water will be removed from the final product in the present invention. In addition, the excipients in the pharmaceutical solid formulations have adhesiveness and allow the materials to be directly compressed or filled into capsules. It can be seen that Therefore, the binder may or may not be added to the solid pharmaceutical composition of the present invention. During the wet granulation, the binder can be added or not be added. If added, based on the total weight of the pharmaceutical composition, the amount of the binder is from 0.1 to 10%, 0.2 to 5%, or 0.5 to 2.5%, and or it may also be used according to the experience of a person skilled in the art.

According to any example of the methods described in the second aspect of the present invention, the lubricant (including glidant) mentioned is used to make the powder material formed, for example, the powder material can be uniformly filled into the capsule shell during encapsulation process. For example, the powder material can be uniformly filled into the mold of the tabletting machine to avoid the sticking to the tablet tooling during compression. Examples of lubricants include but are not limited to: magnesium stearate, calcium stearate, talc, starch, stearic acid, colloidal silica, polyethylene glycol etc. If lubricant is added, the amount of lubricant used can be from 0.1 to 10%, 0.2 to 5%, or 0.2 to 2% relative to the total weight of the pharmaceutical composition, which may also be used according to the experience of a person skilled in the art.

According to any example of the methods described in the second aspect of the present invention, the described solid pharmaceutical composition is an uncoated or coated tablet with a coating material.

According to any example of the methods described in the second aspect of the present invention, the described solid pharmaceutical composition is a coated tablet. Furthermore, the coated tablets comprise a coating material of 1% to 6%, 2% to 5% based on the total weight of the tablet. In one embodiment, the coating material is selected from the group of ethyl cellulose, hydroxypropylmethylcellulose and methacrylic acid-alkyl acrylate copolymers. In one embodiment, the coating material is an aqueous dispersion of hydroxypropylmethylcellulose. In one embodiment, the coating material is Opadry® which is an aqueous dispersion of hydroxypropylmethylcellulose; Furthermore, the coating material may selected from one of the Opadry® 85F20694, Opadry® 85F32004, Opadry® 85F23452 and Opadry®85F18422.

According to any example of the methods described in the second aspect of the present invention, the described solid pharmaceutical composition includes:

Topiramate: 100 parts by weight,

cellulose derivatives: 10 to 100 parts by weight,

lipid substances: 10 to 100 parts by weight

pharmaceutical excipients: 0 to 500 parts by weight.

According to any example of the methods described in the second aspect of the present invention, the described solid pharmaceutical composition includes:

Topiramate: 100 parts by weight,

cellulose derivatives: 15 to 75 parts by weight,

lipid substances: 15 to 75 parts by weight,

The pharmaceutical excipients: 0 to 250 parts by weight.

According to any example of the methods described in the second aspect of the present invention, the described solid pharmaceutical composition includes:

Topiramate: 100 parts by weight,

cellulose derivatives: 20 to 50 parts by weight,

lipid substance: 30 to 50 parts by weight,

pharmaceutical excipients: 0 to 200 parts by weight.

According to any example of the methods described in the second aspect of the present invention, the described solid pharmaceutical composition placed at 40° C. temperature for 5 months, wherein the impurities of 2,3:4,5-di-O-(%) is less than 100%, especially less than 80%, especially less than 70%: for example, from 40 to 100%, from 40% to 80%, or from 40 to 70%.

It is known that the impurity of topiramate and its formulation composition is 2,3,4,5-di-O-(1-methylethylidene)-β-D-fructopyranose (which may be referred to as impurity A in the present invention, English chemical name is: 2,3:4,5-Bis-O-(1-methylethylidene)-β-d-fructopyranose, molecular formula:C₁₂H₂₀O₆, molecular weight:260.28) which requires special attention, especially the changes during the long-term storage process. It is surprisingly found that the use of the cellulose derivatives, lipids and stearic acid or a salt thereof in combination with topiramate that can impart an excellent chemical stability. Especially, the impurity A does not increase with the extending of storage time.

Thus, the third aspect of the present invention provides a method for inhibiting the growth of topiramate impurity of 2,3:4,5-di-O-(1-methylethylidene)-β-D-fructopyranose in solid pharmaceutical compositions of. The method comprises a preparation of the solid pharmaceutical composition including topiramate, cellulose derivative, lipid substance, and altogether.

According to any example of the methods described in the third aspect of the present invention, the described solid pharmaceutical composition comprises topiramate, cellulose derivative, lipid, and optional other pharmaceutical excipients.

According to any example of the methods described in the third aspect of the present invention, the weight ratio of topiramate to the cellulose derivative in the solid pharmaceutical composition is from 100:10 to 100.

According to any example of the methods described in the third aspect of the present invention, the weight ratio of topiramate to the cellulose derivative in the solid pharmaceutical composition is from 100:15 to 75.

According to any example of the methods described in the third aspect of the present invention, the weight ratio of topiramate to the cellulose derivative in the solid pharmaceutical composition is 100:20 to 50.

According to any example of the methods described in the third aspect of the present invention, cellulose derivative in the solid pharmaceutical composition is selected from one of the following materials: methylcellulose, ethyl cellulose, sodium carboxymethylcellulose, hydroxypropyl Cellulose, hydroxyethylcellulose, hydroxypropylmethylcellulose and its combinations thereof. In one embodiment, the cellulose derivative is hydroxypropylmethylcellulose.

According to any example of the methods described in the third aspect of the present invention, the weight ratio of topiramate to the lipids in the solid pharmaceutical composition is 100:10 to 100.

According to any example of the methods described in the third aspect of the present invention, the weight ratio of topiramate to the lipids in the solid pharmaceutical composition is 100:15 to 75.

According to any example of the methods described in the third aspect of the present invention, the weight ratio of topiramate to the lipids in the solid pharmaceutical composition is 100:20 to 50.

According to any example of the methods described in the third aspect of the present invention, the lipids described in the solid pharmaceutical composition refers to a lipophilic substance which includes but is not limited to: C16-C22 fatty acid, carnauba wax, C16-C22 fatty acid glycerides, C16-C22 alkyl alcohols, beeswax, synthetic waxes, hydrogenated vegetable oils and mixtures thereof. In one embodiment, the lipid is selected from:C16-C22 fatty acids, carnauba waxes, C16-C22 fatty acid glycerides, C16-C22 alkyl alcohols and mixtures thereof. In one embodiment, the lipid is a C16-C22 fatty acid glyceride. In one embodiment, the lipid is glyceryl behenate. In one embodiment, the behenate is selected from: monoglycerides, behenic acid diglycerides, behenic acid triglycerides, and mixtures thereof.

According to any example of the methods described in the third aspect of the present invention, the percent weight of topiramate in the described solid pharmaceutical composition is 1 to 99% based on the total weight of the solid pharmaceutical composition, for example 2-75% or 5-50%. In the present invention a lipid substance and a cellulose derivative are used in combination with topiramate to form a solid pharmaceutical composition, and the combination provides an excellent quality. Thus, in addition to the lipid, the cellulose derivatives, and the topiramate, the amount of the other pharmaceutical excipients are not particularly limited and are usually added in a suitable amount to impart a suitable dosage form of the composition. For example, when preparing tablets or capsules, the amount of these pharmaceutical excipients may be adjusted due to the overall weight of the dosage form. For example, when the granules are prepared, the amount of these pharmaceutical excipients may be increased due to overall increased weight of the dosage form.

According to any example of the methods described in the third aspect of the present invention, one or more pharmaceutical excipients in the solid pharmaceutical composition is selected from:filler, disintegrant, binder, lubricant.

According to any example of the methods described in the third aspect of the present invention, the filler (usually also referred to as diluent) in the solid pharmaceutical composition includes but isn't limited to: starch or its derivatives such as corn starch, pregelatinized starch, modified starch,etc.; cellulose or its derivatives such as microcrystalline cellulose, ethyl cellulose, methyl cellulose,etc.; Saccharides such as glucose, sucrose, lactose, mannitol, sorbitol; Neutralized minerals such as calcium carbonate, calcium hydrogen phosphate, and the like, and combinations thereof.

According to any example of the methods described in the third aspect of the present invention, the disintegrant in the described solid pharmaceutical composition includes but not limited to: cross-linked polyethylene polypyrrolidone, sodium starch glycolate, croscarmellose Sodium, low-substituted hydroxypropylcellulose and their combinations. Furthermore, relative to the total weight of the pharmaceutical composition, the pharmaceutical composition comprises 0 to 10%, 0 to 8%, or 0 to 5% of disintegrant, and its amount also can be decided based on the experience of a technical personnel in the field. It's common to use no or use less disintegrant in drug product, such as dissolved-matrix type of sustained-release tablets made for sustained-release purposes. Because the solid pharmaceutical compositions of the present invention may have no disintegrating properties in some cases, for example, encapsulation, the disintegrants may not be added to the pharmaceutical compositions.

According to any example of the methods described in the third aspect of the present invention, the binder in the described solid pharmaceutical composition includes but isn't limited to: polyethylene glycol, starch, polyvinylpyrrolidone, hydroxypropylmethylcellulose, etc, and their combinations. Because the material of many solid pharmaceutical formulations has a certain degree of adhesiveness during the wet granulation process, water can be used as a wetting agent. Water as a wetting agent in the present invention can also be used as a potential binder, although the water added are removed from the final product in the present invention. In addition, the materials used in the solid pharmaceutical formulations have adhesiveness and allow the powder to be directly compressed or filled into capsules. Therefore, the binder may or may not be added to the solid pharmaceutical formulation of the present invention. Even with the wet granulation process, the binder can still not be added. If added, based on the total weight of the pharmaceutical composition, the amount of the binder is 0.1 to 10%, 0.2 to 5%, or 0.5 to 2.5%, and may also be based on the experience of a technical person in the field.

According to any example of the methods described in the third aspect of the present invention, the lubricant (including glidant) in the described solid pharmaceutical composition is used to promote the powder material can be smoothly passing through hopper. For example, the powder material can be uniformly filled into the capsule shell during encapsulation. For example, the powder material can be uniformly filled into the mold of the tabletting machine and the sticking can be prevented during compression process. Examples of lubricants used include, but are not limited to: magnesium stearate, calcium stearate, talc, starch, stearic acid, colloidal silica, polyethylene glycol, etc. If added, the amount of lubricant used relative to the total weight of the pharmaceutical composition is 0.1 to 10%, 0.2 to 5%, or 0.2 to 2%. The amount of lubricant used can also be based on the experience of a technical person in the field.

According to any example of the methods described in the third aspect of the present invention, the solid pharmaceutical composition is a pharmaceutical dosage form selected from the following:tablets, capsules, mini-tablet incapsules, etc.

According to any example of the methods described in the third aspect of the present invention, the described solid pharmaceutical composition is an uncoated or coated tablet with a coating material.

According to any example of the methods described in the third aspect of the present invention, the described solid pharmaceutical composition is a coated tablet. Furthermore, the coated tablets comprise a coating material of 1% to 6%, 2% to 5% based on the total weight of the tablets. In one embodiment, the coating material is selected from:ethyl cellulose, hydroxypropylmethylcellulose, and methacrylic acid-alkyl acrylate copolymers. In one embodiment, the coating material is an aqueous dispersion of hydroxypropylmethylcellulose. In one embodiment, the coating material is Opadry® which is an aqueous dispersion of hydroxypropylmethylcellulose; Furthermore, the coating material is selected from:Opadry® 85F20694, Opadry® 85F32004, Opadry® 85F23452 and Opadry®85F18422.

According to any example of the methods described in the second aspect of the present invention, the described solid pharmaceutical composition includes:

Topiramate: 100 parts by weight,

cellulose derivative: 10 to 100 parts by weight,

lipid substance :10 to 100 parts by weight,

pharmaceutical excipients: 0 to 500 parts by weight.

According to any example of the methods described in the third aspect of the present invention, the solid pharmaceutical composition comprises:

Topiramate: 100 parts by weight,

cellulose derivative: 15 to 75 parts by weight,

lipid substance: 15 to 75 parts by weight,

pharmaceutical excipients: 0 to 250 parts by weight.

According to any example of the methods described in the third aspect of the present invention, the solid pharmaceutical composition comprises:

Topiramate: 100 parts by weight,

cellulose derivative: 20 to 50 parts by weight,

lipid substance: 30 to 50 parts by weight,

pharmaceutical excipients: 0 to 200 parts by weight,

According to any example of the methods described in the third aspect of the present invention, the described solid pharmaceutical compositions placed at 40° C. temperature for 5 months, wherein the impurity of 2,3:4,5-di-O-(1-methylethylidene)-β-D-pyran fructose is less than 100%, especially is less than 80%, especially is less than 70%, for example, 40 to 100%, 40% to 80%, or 40 to 70%.

Furthermore, the fourth aspect of the present invention provides a solid pharmaceutical agent comprising the first pharmaceutical part and the second pharmaceutical part which are independent of each other in physical space.

According to any example of the solid pharmaceutical agents described in the fourth aspect of the present invention, the first pharmaceutical part is a solid pharmaceutical composition described in any one of the first aspect of the present invention.

According to any example of the solid pharmaceutical agents described in the fourth aspect of the present invention, the second pharmaceutical part uses 900 ml of water as the dissolution medium. When the drug dissolution test is carried out at 50 rpm with paddle, the drug released at 45 minutes is more than that 70% of the amount of the drug contained in the second pharmaceutical part.

According to any example of the solid pharmaceutical agents described in the fourth aspect of the present invention, the second pharmaceutical part comprises 10 to 90% of topiramate and 10 to 90% of the pharmaceutical excipients. According to the dissolution performance of the above-mentioned the second part, it is apparent that the pharmaceutical excipients used therein are conventional pharmaceutical excipients other than sustained release pharmaceutical excipients, such as the pharmaceutical excipients described in the solid pharmaceutical compositions according to any examples of the pharmaceutical compositions in the present invention.

According to any example of the solid pharmaceutical agents described in the fourth aspect of the present invention, the weight ratio of topiramate is 1 to 5:1 between the first pharmaceutical part and the second pharmaceutical part.

According to any example of the solid pharmaceutical agents described in the fourth aspect of the present invention, the solid pharmaceuticals agents described in the first pharmaceutical part and the second pharmaceutical part independently exist in the form of bilayer tablets, minitablets in capsules, or pellet in capsules. The term “pellet capsule” is well known in the field as capsules formed by encapsulating the multiple pellets of the same or different types into capsules.

In the invention, any of the technical features of any of the aspects of the present invention or any of the embodiments may also apply to any of the other embodiments or any of the other aspects as long as they are not contradictory. When it's applicable for each other, if necessary, the corresponding features can be properly modified. The various aspects and features of the present invention will be further described below.

All references cited herein are hereby incorporated by reference. If the contents expressed in these documents are inconsistent with the present invention, the contents of the present invention is valid. In addition, the various terms and phrases used in the present invention have the general meaning that well known to a person skilled in the art. Even so, the present invention still wishes to provide a more detailed description and explanation of these terms and phrases, the terms and phrases mentioned. In the case of any inconsistency with the known meaning, the meaning expressed in the present invention shall prevail.

The various aspects of the invention are further described below.

In the present invention, % is a percentage of weight/weight unless otherwise stated.

The topiramate solid pharmaceutical compositions of the present invention can be used for newly diagnosed epilepsy patients as a monotherapy or epilepsy patients switching from a combination therapy to a monotherapy It can also be used as an additional treatment for adult and 2-16 year old children with partial seizures.

The topiramate solid pharmaceutical composition of the present invention is generally used in an amount such that both adults and children are recommended to start treatment from a low dose and then gradually increase the dosage to an effective dose. This product is effective in the treatment of partial seizures in adults and children. In the control plus therapy test, it was confirmed that the concentration of topiramate plasma was not associated with clinical efficacy. There is no evidence that topiramate is intolerant in humans, and dose finding experiments in adults with partial seizures have shown that doses higher than 400 mg/day (600, 800, and 1000 mg/day) do not increase efficacy. Application of this product in the treatment do not have to monitor the topiramate plasma concentration to achieve the best results. During the treatment with this product in combination with phenytoin, only a very small number of cases need to adjust the amount of phenytoin to achieve the best clinical efficacy. The amount of this product may need to be adjusted during the addition or discontinuation phenytoin and carbamazepinein the treatment. It is suitable to take this product with or without food.

During the add-on therapy, for adults (17 years old and above), the recommended dose is to 400 mg/day, divided into 2 doses. Daily dose of 200 mg/day produces lower consistency and efficacy than 400 mg/day. Recommended initial treatment is from 50 mg/day, gradually adjusted to an effective dose. For 2-16 year old children as a concomitant treatment, the recommended total daily dose is 5-9 mg/kg/day, divided into 2 doses. Dosage adjustments should be taken from 25 mg (or less, depending on the dose range of 1-3 mg/kg/day) for the first week aken at night. The the dose is adjusted at every 1 or 2 weeks by increasing 1-3 mg/kg/day (divided into 2 doses) until the best clinical results are reached. The dose adjustment should be based on the clinical results achieved.

For monotherapy, when the other concurrent antiepileptic drug is stopped and the treatment is converted to topiramate therapy alone, withdrawal effect on epilepsy control should be considered. Unless there is safety considerations for the rapid withdrawal of the other antiepileptic drugs, under the normal circumstances, the concurrent medicine should be withdraw slowly. It is recommended to reduce by about ⅓ of the dose every 2 weeks. During the withdrawal of enzyme induction type of medication, topiramate plasma concentration will increase. When the clinical symptom occurs, the topiramate dose should be decreased. Adult (17 years old and above) dose adjustment should start from 25 mg per night for 1 week. Subsequently, the dose is increased to 25-50 mg every week or every 2 weeks twice daily. If the patient is intolerant, adjust the dosage regimen, reduce the dose increment, or prolong the dose adjustment interval. Dosage should be adjusted based on the clinical efficacy. For adult topiramate monotherapy, the recommended daily total dose is 100 mg, and the maximum is 500 mg. Some patients with refractory epilepsy can tolerate up to a daily dose of 1000 mg. The above recommended dose is applicable to all adults, including the elderly and patients without kidney disorders. For children between ages 2 to 16, the dose adjustment should be performed from 0.5-1 mg/kg to start with for 1 week. Then increase by 0.5-1 mg/kg/day (2 doses) for every 1-2 weeks. If the child is intolerant, adjust the dosage regimen, reduce the dose increment, or prolong the dose adjustment interval. The dosage should be adjusted according to the clinical efficacy. For the topiramate monotherapy or single drug treatment, the recommended daily dose is from 3 to 6 mg/kg/day. For children newly diagnosed with partial seizures, the daily dose can be up to 500 mg/day.

### DETAILED DESCRIPTION OF THE INVENTION

The following examples are provided for the purpose of illustration and are not intended to be used in any way and should not be considered as limitations for this invention. A person skilled in the art will recognize the conventional variations and modifications may be made to the following examples without departing from the spirit or scope of the invention.

In the following examples of the preparation of the composition unless otherwise stated, the tablets or capsules are prepared in a batch size of 10 kg. Each tablet or capsule contains 100 mg of the active ingredient listed as 100 mg of topiramate in the formulation.

In the following examples of the preparation of the composition, the various materials are milled by passing through the 80 mesh sieve, unless otherwise stated. In the present invention, various glyceryl behenate and other materials used are readily available in the market. In the following tests of the present invention, when glycerol behenate is used, glycerol dibehenate esters conformed to the British Pharmacopoeia or the European Pharmacopoeia of the version 7.0, unless otherwise specified. As described in the standard, it is a mixture of glyceryl monobeheneate, glyceryl dibehenate, glyceryl tribehenate. Unless otherwise stated, the materials used in the examples, especially the API, are all from the same batch.

### Test Method Example Section

### Test Method Example 1: HPLC Method for Determining the Amount of Active Ingredient

This test method can be used to test the amount of the active ingredient in the in-process samples, and the final product; the details of the examples are as followed:

It is determined according to the Chinese Pharmacopoeia of the 2010 edition, section 2, Appendix IA, the high performance liquid chromatography test method;

The chromatographic conditions and system suitability test:use octyl-bonded silica as a filler; the mobile phase consist of 0.01 mol/L of ammonium acetate solution (adjusted pH to 4.25±0.2 with acetic acid) and acetonitrile (3:1) as mobile phase; the detector is a refractive index detector, the detection temperature and column temperature are 35° C.; theoretical plate numbers for topiramate peak should not be less than 3000;

The test method:accurately weigh the amount of the test sample (equal to topiramate about 60 mg), transfer to a 200 ml volumetric flask, add acetonitrile-water (1:4) of 100.0 ml accurately, sealed and shake for 1 hour, centrifuged, filter the upper clear liquid with 0.2 μm filter, take the filtrate as the test solution; accurately weigh 60 mg of topiramate reference standard, transfer it to a 200 ml volumetric flask, add acetonitrile-water (1:4) 100.0 ml accurately, sealed and shake to dissolve, as the standard solution; inject 50 μl each from sample and standard solution, respectively, into the liquid chromatograph, record the chromatogram, use external standard method to calculate assay value of C12H21NO8S in sample.

### Test Method Example 2: HPLC Method for Determining the Amount of Impurity A in Test Sample

The method can be used to test the amount of the impurity A in active pharmaceutical ingredient, the in-process samples, and the final product, details are as follows:

Test method:accurately weigh the amount of the test sample (equal to topiramate about 120 mg), transfer to a 25 ml-volumetric flask, add acetonitrile-water (1:4) mixture 10.0 ml accurately, sealed and shake for 1 hour, then centrifuged, filter the upper clear liquid with a 0.2 μm filter, take the filtrated solution as the test solution;

Transfer both 1 ml test solution and about 8 mg accurately weighed impurity A reference standard, onto a 200 mL-volumetric flask, add mobile phase to the mark, shake well, then used as the standard solution;

According to the chromatographic conditions in test method example 1, inject accurately measured of 50 μl of sample and standard solution, respectively, into the liquid chromatograph, adjust the sensitivity of the instrument to ensure that the peak area of the topiramate in the control solution could meet the requirement of correct integration. The chromatogram of the test solution should be recorded for double value of the main peak retention time/The external standard method should be used to calculate the value of impurities A with the peak area. Generally speaking, the value of impurity A in topiramate formulation should be less than 0.7%, as required by a person skilled in the art.

### Test Method Example 3: Stability Study

Various test samples were placed in a sealed aluminum-plastic composite film bag to prevent the inside and outside air exchange. The test samples were then placed in a controlled 40° C. stability chamber for 5 months to perform a routine high temperature accelerated stability test.

The assay of the topiramate active ingredient in the samples are determined by using method in test method example 1, including samples in 0-month (not processed at 40° C.) and 5-month (processed at 40° C.) (Unit is mg/g which means the amount of topiramate (mg) in 1 g test sample), and using the following formula to calculate the residual value of topiramate (%) after high temperature treatment in each test sample:

The residual assay (%) of topiramate=[assay in 5-month/assay in 0-month]×100%

The closer to 100% of the above residual assay (%), the more stable the sample is. When the active ingredient is stored for a long time and the content is reduced due to various reasons, the residual assay should be more than 90% after 5 months stored at 40 □ generally. If it is less than 90%, the product is usually considered as unqualified.

The assay of the impurity A in the samples are determined by using method in test method example 2, including samples in 0 month (not processed at 40° C.) and 5 month (processed at 40° C.). The increase in the amount or increment (%) of impurity A in each test sample are calculated based on the following formula:

The increase in the amount (%) of impurity A=[(assay for impurity A in 5 months−assay for impurity A in 0 month)/assay of impurity A in 0 month)]×100%

The closer to 0% value of the above increase amount (%) is, the more stable the sample is; and when the increment is greater, it shows more impurity A in sample.

### Example 1

### Preparation of a Solid Pharmaceutical Combination Comprising Topiramate

According to Sample Number Ex1-01 to Ex1-11 in the following table 1, eleven blends can be prepared with different content of topiramate, glyceryl behenate and hydroxypropylmethylcellulose (HPMC). The preparation of the mixed sample is as follows: (1) Each ingredients was milled to fine powder which can pass 60 mesh sieve. The topiramate, glyceryl behenate and hydroxypropylmethylcellulose were mixed fully, and then divided equally into 2 portions. One portion of these mixture is encapsulated into capsule and the other portion compressed into tablets.

The stability of the tablets from the eleven samples was examined by the method described in Test Method Example 3. The remaining topiramate (%) after 5 months and the increment (%) of the impurity Aare calculated, the result is as follows shown on Table 1:

TABLE 1

| | | glyc- | Hydroxy- | | the |
|---|---|---|---|---|---|
| | | eryl | propyl | the residual | increment |
| Sample | | be- | methyl- | assay of the | of the |
| No. | Topiramate | henate | cellulose | topiramate | impurity A |
| Ex1-01 | 100 | 35 | 0 | 87.2% | 258% |
| Ex1-02 | 100 | 35 | 5 | 95.3% | 206% |
| Ex1-03 | 100 | 35 | 10 | 95.7% | 144% |
| Ex1-04 | 100 | 35 | 20 | 98.4% | 53% |
| Ex1-05 | 100 | 35 | 30 | 98.7% | 45% |
| Ex1-06 | 100 | 35 | 35 | 98.4% | 47% |
| Ex1-07 | 100 | 35 | 40 | 97.6% | 46% |
| Ex1-08 | 100 | 35 | 50 | 96.9% | 38% |
| Ex1-09 | 100 | 35 | 75 | 92.4% | 45% |
| Ex1-10 | 100 | 35 | 100 | 88.5% | 53% |
| Ex1-11 | 100 | 35 | 200 | 84.6% | 47% |

It has been surprisingly shown that when the topiramate and glyceryl behenate are mixed with more than 20 parts of hydroxypropylmethylcellulose and the mixture were stressed by mimicing the high temperature and long term storage condition, the concentration of the impurity A is less than that in the samples contained less HPMC or no HPMC. The rate of impurity A growth is very slow in the sample containing a small amount of hydroxypropylmethylcellulose and slower in the sample containing no HPMC. However, when the amount of hydroxypropylmethylcellulose is too high, for example, when the relative amount of hydroxypropylmethylcellulose is 75 parts by weight or more, the active ingredient degraded rapidly. Therefore, when 20 to 50 parts by weight of hydroxypropylmethylcellulose is incorporated in the case of 100 parts by weight of topiramate with glyceryl behenate, it is not only possible to maintain stable and high level of the active ingredient during long term storage, but also slow growth of impurity.

In addition, the tablets and capsules manufactured from the eleven mixed samples of Ex1-01 to Ex1-11 were analyzed. For each formulation, both the assay of topiramate and the increase of impurity A were reproducible (a difference of no more than 2%).

Comparative Test 11: referring to the formulations of Ex1-04, Ex1-06, Ex1-08 in the Example 1 above, the hydroxypropylmethylcellulose was replaced with the same amount of methylcellulose, hydroxypropylcellulose, or hydroxyethylcellulose, nine samples were obtained and tested in the same manner as in Test Method Example 3 for stability evaluation. The results showed that the assay (%) of topiramate was in the range of 94 to 97%, but the impurity A was increased by more than 180%, between the ranges of 183 to 252%, which indicated that other cellulose derivatives can not inhibit impurity A growth as hydroxypropylmethylcellulose did.

Comparative Test 12: referring to the formulations of Ex1-04, Ex1-06, Ex1-08 in the Example 1 above, the same amount of active ingredient, the methylcellulose, hydroxypropylcellulose, or hydroxyethylcellulose were added to the formulation, nine samples were obtained and tested in the same manner as in Test Method Example 3. The results showed that the assay of topiramate was in the range of 95 to 98%, and the increment for impurities A was less than 62%, both in the range of 40 to 62%, which indicated that the addition of the above-mentioned cellulose derivatives did not affect the action of HPMC.

### Example 2

### Preparation of a Solid Pharmaceutical Combination Comprising Topiramate

According to the table 2, sample number Ex2-01 to Ex2-11, there were eleven blends that were prepared with different content of topiramate, glyceryl behenate, and hydroxypropylmethylcellulose(HPMC). The preparation of the mixed sample was as follows: (1) each material was milled into fine powders that can pass 60 mesh sieves. The formula amount of topiramate, glyceryl behenate and hydroxypropylmethylcellulose were mixed fully to get a powder mixture. Then, the mixture was divided into 2 parts: one part encapsulated into capsule and the other half compressed into tablets.

The 5-month stability of the tablets manufactured from the eleven samples was evaluated by the method described in Test Method Example 3, and the residual assay (%) of the topiramate and the increment (%) of the impurity A were calculated. The results werelisted as follows in Table 2:

TABLE 2

| | | glyc- | hydroxy- | | the |
|---|---|---|---|---|---|
| | | eryl | propyl- | the residual | increment |
| Sample | | be- | methyl- | assay of the | of the |
| No. | Topiramate | henate | cellulose | topiramate | impurity A |
| Ex2-01 | 100 | 35 | 0 | 89.4% | 271% |
| Ex2-02 | 100 | 35 | 5 | 94.5% | 213% |
| Ex2-03 | 100 | 35 | 10 | 96.2% | 136% |
| Ex2-04 | 100 | 35 | 20 | 98.7% | 57% |
| Ex2-05 | 100 | 35 | 30 | 98.3% | 44% |
| Ex2-06 | 100 | 35 | 35 | 98.5% | 48% |
| Ex2-07 | 100 | 35 | 40 | 97.3% | 43% |
| Ex2-08 | 100 | 35 | 50 | 96.4% | 36% |
| Ex2-09 | 100 | 35 | 75 | 91.6% | 43% |
| Ex2-10 | 100 | 35 | 100 | 89.3% | 50% |
| Ex2-11 | 100 | 35 | 200 | 85.7% | 49% |

It has been surprisingly shown that when the topiramate and hydroxypropylmethylcellulose were mixed with equal to or more than 20 parts of glyceryl behenate, the concentration of the impurity A which should be controlled strictly is less, when compared with the mixture contains less or no glyceryl behenate. Hence, the rate of increase of impurity A is slow with a certain amount of glyceryl behenate, far less than samples containing no glyceryl behenate. However, when the amount of glyceryl behenate is too high, for example, when the relative amount of glyceryl behenate is more than 50 parts by weight, the amount of active ingredient decreases rapidly. Therefore, when 20 to 50 parts by weight of glyceryl behenate is incorporated in the mixture of 100 parts by weight of topiramate and in the presence of hydroxypropylmethylcellulose, it is not only possible to maintain a high and stable level of the active ingredient during long-term storage, but also slow impurity growth.

In addition, the tablets and capsules from the eleven mixed samples of Ex2-01 to Ex2-11 were tested. For each formulation, both topiramate assay and the amount of impurity A were reproducible (a difference was no more than 1.5%).

Comparative Test 21: referring to the formulation of Ex2-04, Ex2-06, Ex2-08 in the Example 2 above, the glyceryl behenate was replaced with the same amount of stearic acid, carnauba wax, or stearyl alcohol, there were nine samples obtained. Samples were tested in the same manner as in Test Method Example 3 for stability evaluation. The results showed that topiramate assay is in the range of 87 to 95%, but the impurity A is more than 194% increments, all were in the range of 194 to 242%, which indicates that stearic acid and other similar lipids can not inhibit impurity growth as combination of glyceryl behenate and hydroxypropylmethylcellulose did.

Comparative Test 22: referring to the formulation of Ex2-04, Ex2-06, Ex2-08 in the Example 2 above, the equal amount of stearic acid, carnauba wax, or stearyl alcohol as the active ingredient was additionally added to the formulation, a total of nine samples were obtained and examined as the same manner in Test Method Example 4. The results showed that topiramate assay was in the range of 95 to 98%, and the increment for impurities A was less than 60%, all were within the range of 40 to 60%, which indicated that the addition of the above-mentioned lipids did not affect the effect ofglyceryl behenate.

Comparative Test 23: Four finished products, the market product of topiramate tablets (Chinese Medicine Registration No. H20020557), the topiramate capsule (named as #444 capsules here in this study) based on the formulation and preparation of example 1-3 shown in the instruction of the patent CN1419444A(99803589.0), the pellets (named as #367 pellets here) based on the formulation and preparation of Example 6 (chapter from 0117 to 0127) shown in the instruction from CN102579367B (201210080716.8) and the tablet (named as #501 tablet here) based on the formulation and preparation of example 4 shown in the instruction from CN103417501A (201210162377.8), were placed at the 40 □ condition for 5 months. The data collected shows that assay of topiramate was within the range of 93.4 to 96.7%, which indicated that although it is acceptable, it was not as effective as the present invention; but the increment of the impurities A was in the range of 154 to 232%, which indicated that these products were far less effective than the present invention to slow the increment of the impurity A.

### Example 3

### Preparation of a Solid Pharmaceutical Combination Comprising Topiramate

The pharmaceutical compositions in the present invention were prepared according to the formulation shown in the table 3 below.

TABLE 3

| Ingredient | Weight(mg) | |
|---|---|---|
| Topiramate | 100 | mg |
| Glyceryl behenate | 35 | mg |
| HPMC | Prescribed quantity | |
| Microcrystalline cellulose | 40 | mg |
| Starch | 30 | mg |
| Sodium starch glycolate | 5 | mg |
| PVP K30 | 3 | mg |
| Magnesium stearate | 2 | mg |

Preparation: The ingredients were milled and passed through a 60 mesh sieve. The active pharmaceutical ingredient, glyceryl behenate, and the prescribed amount of hydroxypropylmethylcellulose were mixed evenly. Then, he microcrystalline cellulose and starch were added onto the mixture and mixed evenly. The above mixed powder was wet granulated with a 5% PVP K30 solution as a binder which prepared by using 50% ethanol to get soft material. Then, the wet granules were dried at 50° C. until the moisture content was less than 2.5%. The dry granules were mixed with the disintegrant and the lubricant evenly and divided in to 2 parts: half of the final blended material encapsulated into the hard capsule shell and the other half compressed into tablets.

According to the formulation in Table 3, hydroxypropylmethylcellulose was mixed with the amounts (parts by weight) of the topiramate and hydroxypropylmethylcellulose described in Sample of No. Ex3-01 to Ex3-07 listed in Table 4 below. Seven samples of tablets and capsules were obtained.

The stability of these seven samples was tested by the method in Test Method Example 3 for stability evaluation. The residual assay (%) of topiramate and the increment (%) of impurities A after 5 monthswere calculated. The results are shown in Table 4:

TABLE 4

| | | glyc- | | | the |
|---|---|---|---|---|---|
| | | eryl | | the residual | increment |
| Sample | | be- | | assay of the | of the |
| No. | Topiramate | henate | HPMC | topiramate | impurity A |
| Ex3-01 | 100 | 35 | 0 | 83.5% | 264% |
| Ex3-02 | 100 | 35 | 5 | 87.9% | 213% |
| Ex3-03 | 100 | 35 | 20 | 96.4% | 63% |
| Ex3-04 | 100 | 35 | 35 | 98.3% | 68% |
| Ex3-05 | 100 | 35 | 50 | 95.9% | 54% |
| Ex3-06 | 100 | 35 | 75 | 91.2% | 67% |
| Ex3-07 | 100 | 35 | 100 | 86.5% | 65% |

The results show that even if other conventional excipients were added, these tablets still had a typical correlation with the amount of hydroxypropylmethylcellulose added in both the residual assay (%) of topiramate and the increment (%) of impurity A. Both these two parameters were not acceptable when the samples had less or no HPMC (<20 parts by weight), and when the amount of hydroxypropylmethylcellulose was too high (>50 parts by weight), the active ingredient assay was still not acceptable. When 20 to 50 parts by weight of hydroxypropylmethylcellulose was incorporated with respect to 100 parts by weight of topiramate, not only the active ingredient can be maintained at a high stable level during the long term storage, but also the impurity growth is slow.

### Example 4

### Preparation of a Solid Pharmaceutical Combination Comprising Topiramate

The pharmaceutical compositions in the present invention were prepared according to the formulation shown in the table 5 below.

TABLE 5

| Ingredient | Weight(mg) | |
|---|---|---|
| Topiramate | 100 | mg |
| Glyceryl behenate | Prescribed quantity | |
| HPMC | 35 | mg |
| Microcrystalline cellulose | 40 | mg |
| Starch | 30 | mg |
| Sodium starch glycolate | 5 | mg |
| PVP K30 | 3 | mg |
| Magnesium stearate | 2 | mg |

Preparation: The materials were milled and passed through a 60 mesh sieve. The active pharmaceutical ingredient, hydroxypropylmethylcellulose, and the prescribed amount of glyceryl behenate were mixed evenly. Then, microcrystalline cellulose as filler with starch was added and mixed evenly. The above mixed powder is wet granulated with a 5% PVP K30 solution as a binder which prepared by using 50% ethanol to get soft material. Then the wet granules were dried at 50° C. until the moisture content was less than 2.5%. The dry granules were mixed with the disintegrant and the lubricant evenly, and divided in to 2 parts: half of the final blended materials encapsulated into the hard capsule shell and the other half compressed into tablets.

According to the formulation in Table 6, glyceryl behenate was mixed with the amount of topiramate and glyceryl behenate described in Sample No. Ex4-01 to Ex4-07 listed below, and the seven mixture samples of tablets and capsules were obtained.

The stability of these seven samples was tested by the method of Test Method Example 4 for stability evaluation. The residual assay (%) of topiramate and the increment (%) of impurities A after 6 months were calculated. The results are shown in Table 6:

TABLE 6

| | | glyc- | | | the |
|---|---|---|---|---|---|
| | | eryl | | the residual | increment |
| Sample | | be- | | assay of the | of the |
| No. | Topiramate | henate | HPMC | topiramate | impurity A |
| Ex4-01 | 100 | 35 | 0 | 93.5% | 242% |
| Ex4-02 | 100 | 35 | 5 | 94.4% | 194% |
| Ex4-03 | 100 | 35 | 20 | 96.3% | 66% |
| Ex4-04 | 100 | 35 | 35 | 96.7% | 55% |
| Ex4-05 | 100 | 35 | 50 | 96.3% | 59% |
| Ex4-06 | 100 | 35 | 75 | 92.3% | 64% |
| Ex4-07 | 100 | 35 | 100 | 87.2% | 56% |

The results show that even if other conventional excipients were added, these tablets still had a typical correlation with the amount of glyceryl behenate added in both the residual assay (%) of topiramate and the increment (%) of impurity A. Both these two parameters were not acceptable when the samples had less or no HPMC (<20 parts by weight) added, and when the amount of glyceryl behenate was too high (>50 parts by weight), the active ingredient assay was still not acceptable. When 20 to 50 parts by weight of glyceryl behenate was incorporated with respect to 100 parts by weight of topiramate, not only the active ingredient can be maintained at a high stable level during the long term storage, but also the impurity growth is slow.

### Example 5

### Preparation of a Solid Pharmaceutical Combination Comprising Topiramate

| Ingredient | Weight(mg) | |
|---|---|---|
| Topiramate | 100 | mg |
| Glyceryl behenate | 35 | mg |
| HPMC | 35 | mg |
| Microcrystalline cellulose (filler) | 100 | mg |
| PVP(binder, use 50% alcohol to get a solution | 5 | mg |
| with 5% concentration PVP) | | |
| Croscarmellose sodium (disintegrant) | 8 | mg |
| PEG6000(lubricant) | 2 | mg |

Preparation: The materials were milled and passed through 60 mesh sieve. The active pharmaceutical ingredient, glyceryl behenate, and hydroxypropylmethylcellulose were mixed evenly. Then, the filler was added and mix evenly. The above blend was granulated with binder and dried at 50° C. until the moisture content was less than 2.5%. The granules were remixed with the disintegrant and the lubricant and divided into 2 parts: half of the blend materials encapsulated into the hard capsule shell and the other half compressed into tablets. Half of the compressed tablets was directly sealed and packaged as core tablets; and the other half of tablets were coated with Opadry® 85F20694 with the coating material account for 3% of the total weight of the final tablets.

The capsules, core tablets and coated tablets obtained in this example were sealed and tested for stability evaluation by the method in Test Method 3. The results showed that the residual assay of topiramate in capsules, core tablets and coated tablets was within the range of 97.3˜98.2%, and the increment of impurity A was within the range of 45˜60%, which indicated that these preparations have good chemical stability.

### Example 6

### Preparation of a Solid Pharmaceutical Combination Comprising Topiramate

| Ingredient | Weight(mg) | |
|---|---|---|
| Topiramate | 100 | mg |
| Glyceryl behenate | 20 | mg |
| HPMC | 50 | mg |
| Microcrystalline cellulose (filler) | 40 | mg |
| Corn starch (filler) | 20 | mg |
| PEG2000(binder, use water to get a solution with | 5 | mg |
| 5% concentration PEG2000) | | |
| Crosslinked Polyvinylpyrrolidone (disintegrant) | 8 | mg |
| Colloidal silica (lubricant) | 2 | mg |
| Magnesium stearate (lubricant) | 1 | mg |

Preparation: The materials were milled and passed through 60 mesh sieve. The active pharmaceutical ingredient, glyceryl behenate, and hydroxypropylmethylcellulose were mixed evenly. Then the filler was added and mixed evenly. The above mixed powder was wet granulated with a binder. Then, the granules were dried at 50° C. until the moisture content was less than 2.5%. The resulting dry granules were mixed with the disintegrant and the lubricant, and divided into 2 parts: half of the blend encapsulated into the hard capsule shell and the other half compressed into tablets. Half of the resulting tablets were directly sealed and packaged as a core tablet and the other half of the tablets were coated with Opadry®85F23452 with the coating material account for 3% of the total weight of the final tablets.

The capsules, core tablets, and coated tablets obtained in this example were sealed and tested for stability evaluation by the method of Test Method 3. The results showed that assay of topiramate in capsules, core tablets, and coated tablets were in the range of 96.5˜98.2%, and the increment of impurity A were in the range of 45˜55%, which indicated thatthepreparations have good chemical stability.

### Example 7

### Preparation of a Solid Pharmaceutical Combination Comprising Topiramate

| Ingredient | Weight(mg) | |
|---|---|---|
| Topiramate | 100 | mg |
| Glyceryl behenate | 50 | mg |
| HPMC | 20 | mg |
| Corn starch(filler) | 60 | mg |
| Water(binder) | Appropriate amount | |
| Lowly substituted hydroxypropylcellulose | 5 | mg |
| (disintegrant) | | |
| Magnesium stearate (lubricant) | 2 | mg |

Preparation: The materials were milled and passed through 60 mesh sieve. The active pharmaceutical ingredient, glyceryl behenate, and hydroxypropylmethylcellulose were mixed evenly. Then, the filler was added and mixed evenly. The above mixed powder was wet granulated with a binder. The wet granules were dried at 50° C. until the moisture content was less than 2.5%. The dry granules were mixed with the disintegrant and the lubricant, and divided into 2 parts: half of the material encapsulated into the hard capsule shells and the other half of compressed into tablets. Half of the tablets were then directly sealed and packaged as core tablets; and the other half of the tablets were coated with Opadry®85F32004 with the coating material account for 3% of the total weight of the final tablets.

The capsules, core tablets, and coated tablets obtained in this example were sealed and tested for stability evaluation by the method of Test Method 3. The results showed that assay of topiramate in capsules, core tablets, and coated tablets were within the range of 96.5˜97.8%, and the increment of impurity A were within the range of 44˜56%, which indicated that these preparations have good chemical stability.

In addition, referring to the formulation and preparation in the example 7 above, the glyceryl behenate is replaced by an equal amount of glyceryl behenate with monoester content more than 95%, glyceryl behenate with diester content more than 95%, or glyceryl behenate with triglyceride content more than 95%, to obtain three kinds of coated tablets which were numbered as Ex71, Ex72, Ex73 respectively. The three kinds of coated tablets were sealed and tested for stability evaluation according to the method of test method 4. The results showed that the residual assay of topiramate in these three coated tablets were all in the range of 96.6˜97.7%, and the increment of impurity A were all in the range of 43˜55%, which indicated that these preparations have good chemical stability.

### Example 8

### Investigation of Drug Properties

The tablets prepared from Ex1-06, Ex2-06, Ex3-04, Ex4-04, Example 5, Example 6, Example 7 were tested for dissolution, using 900 ml of water as the release medium with paddle at 50 rpm. The results showed that the release amount of the active from the tablets were in the range of 15 to 45% at 1 hour, and the release amount were in the range of 30 to 60% at 4 hours, and the release amount were in the range of 50 to 80% at 8 hours, and the release amount were in the range of 70 to 100% at 12 hours. This indicated that the solid pharmaceutical compositions of the present invention were capable of exhibiting sustained release characteristics.

### Example 9

### A Combination of a Medication That Has a Conventional Release Performance and a Medication That Has a Sustained Release Performance

Formulation 5a: According to the formulation and preparation of Example 5, the formulation was compressed into a tablet containing topiramate 20 mg and this tablet has a sustained release property.

Formulation 5b: According to the formulation and preparation of Example 5, glyceryl behenate and hydroxypropylmethylcellulose were not added. The formulation was compressed into tablets containing topiramate 20 mg, and the tablets had an immediate release property. That is, when using 900 ml of water as the release medium and paddling at 50 rpm speed to determine drug dissolution, more than 78% of the drug was released at 45 minutes.

Four tablets obtained from Formulation 5a and one tablet obtained from Formulation 5b were encapsulated into a hard capsule shell as a solid formulation having the characteristics described in any example of the solid pharmaceutical agents described in the fourth aspect of the present invention.

### Example 10

### A Combination of a Medication That Has a Conventional Release Property and a Medication That Has a Sustained Release Property

Formulation 6a: According to the formulation and preparation of Example 6, the formulation was compressed into a tablet containing topiramate 20 mg and this tablet has a sustained release property.

Formulation 6b: According to the formulation and preparation of Example 6, glyceryl behenate and hydroxypropylmethylcellulose were not added. The formulation was compressed into tablets containing topiramate 20 mg and the tablets had an immediate release property. That is, when using 900 ml of water as the release medium and paddling at 50 rpm speed to determine drug dissolution, more than 73% of the drug was released at 45 minutes.

Two tablets obtained from Formulation 6a and two tablets obtained from Formulation 6b were encapsulated into a hard capsule shell as a solid formulation having the characteristics described in any one example of the fourth aspect of the present invention.

### Example 11

### A Combination of a Medication That Had a Conventional Release Performance and a Medication That Had a Delayed Release Performance

Formulation 7a: According to the formulation and preparation of Example 7, the formulation was compressed into a tablet containing topiramate 20 mg and this tablet has a sustained release property.

Formulation 7b: According to the formulation and preparation of Example 7, glyceryl behenate and hydroxypropylmethylcellulose were not added. The formulation was compressed into tablets containing topiramate 20 mg, and the tablets had an immediate release property. That is, when using 900 ml of water as the release medium and paddling at 50 rpm speed to determine drug dissolution, more than 78% of the drug was released at 45 minutes.

The five tablets obtained from Formulation 7a and one tablet obtained from Formulation 7b are encapsulated into a hard capsule shell as a solid formulation having the characteristics described in any example of the solid pharmaceutical agents described in the fourth aspect of the present invention.

For Examples 9 to 11, the immediate release tablets and the sustained release tablets may be made into pellets, respectively. The pellets of these two release properties may be encapsulated into hard capsule shells in proportion as a solid formulation having the characteristics described in any example of the solid pharmaceutical agents described in the fourth aspect of the present invention.

In addition, for Examples from 9 to 11, it was also possible to compress the immediate release portion and the sustained release portion into a double layer tablet using a bi-layer tablet press, and similarly obtain a composition having the characteristics described in any example of the solid pharmaceutical agents described in the fourth aspect of the present invention.

### INDUSTRY APPLICABILITY

The present invention provides a pharmaceutical composition comprising topiramate. The topiramate solid pharmaceutical composition of the present invention can be used as monotherapy for patients newly diagnosed with epilepsy or for epilepsy patients who have been previously treated with combination agents, and it can also be used for adult and for children of 2-16 year old as an add-on treatment for partial seizures.

1-68. (canceled)

69. A solid pharmaceutical composition comprising topiramate, a cellulose derivative, a lipid and optional pharmaceutical excipients; said cellulose derivative comprising hydroxypropylmethylcellulose, said lipid comprising a glyceryl behenate; wherein the weight ratio of topiramate to hydroxypropylmethylcellulose and glyceryl behenate is from 100:15 to 75:15 to 75.

70. The solid pharmaceutical composition according to claim 69, wherein the weight ratio of topiramate to hydroxypropylmethylcellulose is 100:20 to 50, and the weight ratio of topiramate to glyceryl behenate is 100:20 to 50.

71. The solid pharmaceutical composition according to claim 69, wherein said cellulose derivative further comprises a material selected from the group consisting of methylcellulose, ethylcellulose, sodium carboxymethylcellulose, hydroxypropylcellulose, hydroxyl ethylcellulose, hydroxypropylmethylcellulose and combinations thereof; and/or said lipid substance further comprises a material selected from the group consisting of C16-C22 fatty acid, carnauba wax, C16-C22 fatty acid glyceride, C16-C22 alkyl alcohol, beeswax, Synthetic waxes, hydrogenated vegetable oils and mixtures thereof.

72. The solid pharmaceutical composition according to claim 69, wherein the glyceryl behenate is selected from the group consisting of behenic acid monoglycerides, behenic acid diglycerides, behenic acid triglycerides, and mixtures thereof.

73. The solid pharmaceutical composition according to claim 69, wherein the topiramate accounts for 2 to 75% of the total weight of the solid pharmaceutical composition.

74. The solid pharmaceutical composition according to claim 69, which is one or more selected from the group consisting of a filler, a disintegrant, a binder and a lubricant.

75. The solid pharmaceutical composition according to claim 74, wherein the filler is selected from the group consisting of starch or its derivatives, corn starch, pregelatinized starch, modified starch, cellulose or its derivatives, microcrystalline cellulose, ethyl cellulose, methyl cellulose; carbohydrates, glucose, sucrose, lactose, mannitol, sorbitol; neutral minerals, calcium carbonate, calcium phosphate and combinations thereof; the disintegrant is selected from the group consisting of crosslinked polyvinylpyrrolidone, sodium starch glycolate, croscarmellose sodium, low-substituted hydroxypropylcellulose and combinations thereof; the binder is selected from the group consisting of polyethylene glycol, starch, polyvinylpyrrolidone, hydroxypropylmethylcellulose and the like, and combinations thereof; the lubricant is selected from the group consisting of magnesium stearate, calcium stearate, talc, starch, stearic acid, colloidal silica, and polyethylene glycol.

76. The solid pharmaceutical composition according to claim 69, which is a pharmaceutical dosage form selected from the group consisting of tablets, capsules and mini-tablet in capsules.

77. The solid pharmaceutical composition according to claim 69, which is a coated tablet comprising a coating material of 1% to 6% by weight based on the total weight of the tablet.

78. The solid pharmaceutical composition according to claim 77, wherein said coating material is selected from the group consisting of ethylcellulose, hydroxypropylmethylcellulose and methacrylic acid-alkyl acrylate copolymers.

79. The solid pharmaceutical composition according to claim 77, wherein said coating material is an aqueous dispersion of hydroxypropylmethylcellulose.

80. The solid pharmaceutical composition according to claim 69, which comprises 100 parts by weight of topiramate, 10 to 100 parts by weight of a cellulose derivative, 10 to 100 parts by weight of a lipid substance and 0 to 500 parts by weight of a pharmaceutically acceptable excipients.

81. The solid pharmaceutical composition according to claim 69, which comprises 100 parts by weight of topiramate, 15 to 75 parts by weight of cellulose derivative, 15 to 75 parts by weight of lipid substance and 0 to 250 parts by weight of pharmaceutically acceptable excipient.

82. The solid pharmaceutical composition according to claim 69, which is left at a temperature of 40° C. for 5 months, wherein the increment (%) of impurities 2,3:4,5-di-O-(1-methylethylidene)-β-D-fructose before and after high temperature treatment is less than 70%, for example, 40 to 70%.
